(19) United States
(12) Patent Application Publication
(10) Pub. No.: US 2009/0148417 A1
(43) Pub. Date: Jun. 11, 2009

(54) CARBON NANOTUBES SERVING AS STEM CELL SCAFFOLD

(75) Inventors: Sung Su Kim, Gyeonggi-do (KR); Yoo Hun Noh, Seoul (KR); Ok Ja Yoon, Seoul (KR); Seung Hoon Yoo, Seoul (KR)

Correspondence Address: BUCHANAN, INGERSOLL & ROONEY PC, POST OFFICE BOX 1404, ALEXANDRIA, VA, 22313-1404, US

(73) Assignee: BRAINGUARD CO., LTD, SEOUL (KR)

(21) Appl. No.: 11/989,475

(22) PCT Filed: Jul. 27, 2006
(86) PCT No.: PCT/KR2006/002963

§ 371 Date: Jan. 25, 2008

(30) Foreign Application Priority Data
Jul. 28, 2005 (KR) 10-2005-0068912

(51) Int. Cl. A61K 35/12; A61P 9/10; A61P 25/00

(52) U.S. Cl. 424/93.7; 977/742

(57) ABSTRACT

The present invention relates to a scaffold for transplanting a stem cell comprising a carbon nanotube without cytotoxicity, and a composition for stem cell therapy comprising (a) a stem cell; and (b) a carbon nanotube serving as a stem cell scaffold without cytotoxicity. The scaffold for transplanting stem cells comprising carbon nanotubes show excellent effects without cytotoxicity on networking between differentiated stem cells and tissues present in transplantation sites, thereby showing significant cell therapy efficacy.

### FIELD OF THE INVENTION

The present invention relates to a scaffold for transplanting a stem cell and a composition for stem cell therapy.

### DESCRIPTION OF THE RELATED ART

Multipotent stem cells have become highlighted as therapeutic agents for ischemia, Parkinson's disease, Alzheimer's disease, cardiac infarction, and coronaria and liver diseases.

Stem cells transplanted become effective in treatment of diseases only if the following requirements are met: The first requirement is that stem cells transplanted are differentiated into a cell type of interest. The second requirement is to form networks between differentiated stem cells and surrounding tissues and cells.

However, in the practical realm, stem cells transplanted in injured region are very likely to be washed away with no formation of networks (e.g., neuron networks). To maker matters worse, they are delivered to undesirable region and differentiated into undesirable cell types.

Accordingly, there have been made extensive researches to develop scaffolds without in vivo toxicities for stem cell. However, the development of scaffolds without toxicities has been considered difficult tasks and the injection of scaffolds into body has been frequently reported to cause adverse effects. Therefore, there remains a need to develop a novel scaffold having convenient clinical applicability with no in vivo toxicities.

Throughout this application, several patents and publications are referenced and citations are provided in parentheses. The disclosure of these patents and publications is incorporated into this application in order to more fully describe this invention and the state of the art to which this invention pertains.

### DETAILED DESCRIPTION OF THIS INVENTION

The present inventors have made extensive researches to meet a need in the art and as a result, the present inventors have discovered that carbon nanotubes show excellent effects without cytotoxicity on networking between differentiated stem cells and tissues present in transplantation sites, thereby showing significant cell therapy efficacy.

Accordingly, it is an object of this invention to provide a non-cytotoxic scaffold for transplanting a stem cell.

It is another object of this invention to provide a composition for stem cell therapy exhibiting improved therapeutic efficacy.

It is still another object of this invention to provide a cell therapy method using a stem cell.

It is further object of this invention to provide a use of carbon nanotubes for manufacturing a medicament for cell therapy.

Other objects and advantages of the present invention will become apparent from the detailed description to follow taken in conjugation with the appended claims and drawings.

In one aspect of this invention, there is provided a scaffold for transplanting a stem cell, which comprises a carbon nanotube; wherein the scaffold does not exhibit cytotoxicity.

In another aspect of this invention, there is provided a composition for stem cell therapy, which comprises: (a) a stem cell; and (b) a carbon nanotube serving as a stem cell scaffold without cytotoxicity.

In still another aspect of this invention, there is provided a cell therapy method using a stem cell, which comprises administering to an animal a composition for stem cell therapy comprising (a) a stem cell; and (b) a carbon nanotube serving as a stem cell scaffold without cytotoxicity.

In further aspect of this invention, there is provided a novel use of a composition comprising (a) a stem cell; and (b) a carbon nanotube serving as a stem cell scaffold without cytotoxicity for manufacturing a medicament for cell therapy.

The present inventors have made extensive researches to overcome problems associated with forming networks between differentiated stem cells and tissues present in a transplantation site, considered as significant obstacles in stem cell therapy. As a result, the present inventors have discovered that carbon nanotubes show excellent effects without cytotoxicity on networking between differentiated stem cells and tissues present in transplantation sites, thereby showing significant cell therapy efficacy.

Carbon nanotubes serving as stem cell scaffold used in this invention is one of fullerenes, carbon-cage molecule group. Fullerenes may have the form of sphere (buckyball) or tube (nanotube).

The term used herein “carbon nanotube (CNT)” means a carbon-cage molecule structure, including fullerene, carbon buckyball and carbon nanotube, preferably, carbon nanotube. Carbon nanotubes are divided to carbon nanofiber and carbon nanoparticle based on aggregation. Carbon nanotubes may exist as multilayered shell, multi-wall nanotube or single-wall nanotube. Preferably, carbon nanotubes used in this invention are single-wall nanotube. According to a preferred embodiment, carbon nanotubes are functionalized CNT. The functional groups linked to CNT includes thiol and carboxyl group. The functionalized CNT allows for the decrease in aggregation between CNT molecules. The carbon number of CNT used in this invention is not limited, preferably, C₂₀-C₁₅₀. Carbon nanotubes used in this invention may be prepared in accordance with various processes known in the art (see U.S. Pat. Nos. 5,753,088, 5,641,466, 5,292,813 and 5,558,903).

CNT, an advanced material for nanotechnology, firstly discovered in the year of 1991 was researched for its novel uses and functions, its applications have been widened. CNT generally exhibits high mechanical strength, and can be manipulated to have electrically conductive property. Furthermore, CNT has hydrophilic as well as lipophilic properties (Mattson M P, et al., J Mol Neurosci 14(3):175-82, 2000), and has been known to provide a pathway for passing various molecules (Nadine Wong Shi Kam et al, JACS 126:6850-6851, 2004; Nadine Wong Shi Kam et al., JACS 127:6021-6026, 2005). In addition, some researchers have made research to develop drug delivery systems using CNT (Zhu Yinghuai, et al., JACS 127:9875-9880, 2005). Such features and characteristics described above permit CNT to actively be researched. However, applications of CNT to human body have not yet been extensively studied.

The present invention is firstly to provide a novel use of CNT as stem cell scaffold.

The scaffold for transplanting stem cell comprising CNT shows excellent scaffold properties in networking between differentiated stem cells and surrounding cells. As demonstrated in Examples, the CNT stem cell scaffold exhibits considerable cell adhesiveness to improve cell density and cell-to cell adhesion, and no cytotoxicity. Such feature of CNT contributes to the formation of networking between stem cells transplanted and surrounding tissues, allowing stem cells transplanted to exert their functions and effects. Furthermore, such feature prevents stem cells transplanted to be washed away.

In particular, unlikely to conventional scaffolds, the scaffold for transplanting stem cell comprising CNT is easily transplanted as carbon nanoparticles with stem cells using syringe. Furthermore, the scaffold of this invention promotes electric/physiological actions of cells unlike to silicone. In addition to this, CNT used in this invention has some advantages in the senses that it reduces inflammatory responses (Shvedova A A, et al., Am. J. Physiol Lung Cell Mol Physiol, in print, 2005).

Since CNT is well mixed with stem cells and injected into sites of interest, it can decrease adverse effects associated with operation. CNT molecules injected form structures over time and the structures are suitable in the formation of cell-to-cell networks. The electric conductivity of CNT permits it to be delivered to sites of interest via electric induction, thereby making it possible to serve as stem cell scaffolds at sites with disrupted tissues.

Stem cells applied to this invention are not restricted, including any stem cell having inherent characteristics such as non-differentiation, infinite proliferation and differentiative potential to specific cells. The preferable stem cells used in this invention are classified into two groups: pluripotent stem cells such as embryonic stem cell and embryonic germ cell; and multipotent stem cells. Embryonic stem cells are derived from inner cell mass of blastocyst, and embryonic germ cells are derived from primordial germ cells present in 5-10 week aged gonadal ridge. Multipotent stem cells are found in embryonic tissues, fetus tissues or adult tissues, including adult stem cells. Pluripotent stem cells are indefinitely proliferated in vitro and differentiate to three germ layers (ectoderm, mesoderm and endoderm). Unlikely, multipotent stem cells have capability to differentiate to their precursor tissues and their self-renewal potency is restricted. The source of multipotent stem cells includes any type of tissues, in particular, bone marrow, blood, liver, skin, intestine, spleen, brain, skeletal muscle and dental pulp.

Preferably, stem cells used in this invention are embryonic stem cell, adult stem cell, embryonic germ cell and embryonic carcinoma cell, more preferably, embryonic stem cell and adult stem cell.

Preferably, the cell therapy composition of this invention further comprises inducers for stem cell differentiation. Preferable example of the inducer comprises retinoic acid, ascorbic acid, melatonine and various growth factors [e.g., GDNF (glial cell line-derived neurotrophic factor), EGF (epidermal growth factor), NGF (nerve growth factor)].

The diseases or disorders treated by the present composition comprise all diseases or disorders known to be treated by stem cell therapy. Preferably, the cell therapy composition of this invention is applied to the treatment of neuronal diseases, cardiac infarction, injury of spinal column and degenerative rhinitis.

According to a preferred embodiment, the stem cell contained in this invention is neuronal stem cell and the composition is one for treating neuronal diseases.

Where the cell therapy composition is used to treat neuronal diseases, the diseases includes any neuronal diseases caused by damage of neuronal cells. Preferably, the neuronal disease is selected from the group consisting of neurodegenerative disorder and ischemia-reperfusion injury. More preferably, the neurodegenerative disorder is selected from the group consisting of Alzheimer's disease, Huntington's disease, Parkinson's disease and amyotrophic lateral sclerosis, most preferably, Parkinson's disease. According to a preferred embodiment, the ischemia-reperfusion injury is ischemic stroke.

The scaffold for transplanting stem cell comprising CNT is easily transplanted as carbon nanoparticles with stem cells using syringe. Therefore, it is preferable that the cell therapy composition of this invention comprises carbon nanotubes in the form of suspension of carbon nanoparticles.

The suitable amount of CNT in the cell therapy composition is in the range of 0.002-10 mg/ml, preferably, 0.01-1 mg/ml, more preferably, 0.01-0.5 mg/ml, and most preferably 0.01-0.3 mg/ml.

In the cell therapy compositions of this invention, the pharmaceutically acceptable carrier may be conventional one for formulation, including carbohydrates (e.g., lactose, amylose, dextrose, sucrose, sorbitol, mannitol, starch, cellulose), gum acacia, calcium phosphate, alginate, gelatin, calcium silicate, microcrystalline cellulose, polyvinylpyrrolidone, water, salt solutions, alcohols, gum arabic, syrup, vegetable oils (e.g., corn oil, cotton-seed oil, peanut oil, olive oil, coconut oil), polyethylene glycols, methyl cellulose, methylhydroxy benzoate, propylhydroxy benzoate, talc, magnesium stearate and mineral oil, but not limited to. The pharmaceutical compositions of this invention, further may contain wetting agent, sweetening agent, emulsifier, buffer, suspending agent, preservatives, flavors, perfumes, lubricant, stabilizer, or mixtures of these substances. Details of suitable pharmaceutically acceptable carriers and formulations can be found in Remington's Pharmaceutical Sciences(19th ed., 1995), which is incorporated herein by reference.

The cell therapy composition of this invention may be parenterally administered, most preferably by local injection.

The correct dosage of the pharmaceutical compositions of this invention will be varied according to the particular formulation, the mode of application, age, body weight and gender of the patient, diet, time of administration, route of administration, condition of the patient, excretion rate, reaction sensitivity and so on. Preferably, the unit dosage of the pharmaceutical compositions of this invention is 2×10⁵−2×10⁶ cells, generally injected once or twice.

According to conventional techniques known to those skilled in the art, the cell therapy compositions of this invention can be formulated with pharmaceutical acceptable carrier and/or vehicle, finally providing several forms including a unit dosage form or a multi-unit dosage forms. The dosage forms can comprise a solution, a suspension or a emulsion in an oily or aqueous medium as well as further dispersions or stabilizers.

The cell therapy composition of this invention promotes the formation of networking between stem cells transplanted and surrounding tissues, allowing stem cells transplanted to fully exert their functions and effects.

The following specific examples are intended to be illustrative of the invention and should not be construed as limiting the scope of the invention as defined by appended claims.

### BRIEF DESCRIPTION OF THE DRAWINGS

FIG. 1 is an image of electron microscope demonstrating adhesive property of carbon nanotubes (CNT) to P19 EC stem cells.

FIG. 2 shows a graph representing the influence of CNT on cell-to-cell adhesion.

FIGS. 3a-3c are graphs representing no in vitro toxicities of CNT. FIGS. 3a-3c correspond to the results of functionalized Single-Walled CNT (f-SWCNT), functionalized Multi-Walled CNT (f-MWCNT) and CNT fiber, respectively.

FIG. 3d represents PI and FDA staining results verifying whether CNT has in vitro toxicities.

FIG. 4 represents no induction of inflammatory reactions by CNT.

FIG. 5a is an image of DCF-DA staining to examine the generation of reactive oxygen species by CNT.

FIG. 5b represents fluoremeter results to analyze the generation of reactive oxygen species by CNT.

FIG. 6 is a graph representing no in vivo toxicity of CNT.

FIG. 7 demonstrates the therapeutic efficacy of mixtures of CNT and stem cells in Parkinson's animal model.

FIG. 8a demonstrates the memory recovery caused by mixtures of CNT and stem cells in ischemia animal model.

FIG. 8b is a graph representing results of passive avoidance test using mixtures of CNT and stem cells in ischemia animal model.

FIG. 9 demonstrates the improvement in cognitive function by mixtures of CNT and stem cells in Alzheimer's animal model.

### EXAMPLES

### Materials and Methods

### Cell Culture

SK-NSH (ATCC) was used as neuronal cells and cultured in DMEM (GIBCO) supplemented with 10% fetal bovine serum (FBS, GIBCO). P19 EC (embryonic carcinoma, Cell Bank, Korea), a subclone of P19 mouse embryonal carcinoma cell line was used as stem cells and cultured in α-MEM (GIBCO) supplemented with 7.5% Calf serum (CS, GIBCO) and 2.5% FBS (GIBCO). In addition, astrocyte A172 (ATCC) was used for experiments and cultured in the same culture as SKN-SH. Using cells, cytotoxicity, cell adhesion and structure-forming potential of CNT were experimented.

### Carbon Nanotubes

Three types of carbon nanotubes, functionalized Single-Walled CNT (f-SWCNT), functionalized Multi-Walled CNT (f-MWCNT) and CNT nanofiber were used. The characteristics of carbon nanotubes are summarized in Table 1. f-SWCNT and f-MWCNT were functionalized with carboxyl group.

TABLE 1

| Items | f-SWCNT | f-MWCNT | Carbon nanofiber |
|---|---|---|---|
| Diameter (nm) | 0.6-2.1 nm | 15-50 nm | 50-170 nm |
| Length (μm) | — | 10-20 μm | 10-20 μm |
| D₀₀₂ (Å) | — | 3.49 | 3.31 |
| Specific surface area | 595 | 49 | 21 |
| (M²/g) | | | |
| Density (g/cm³) | — | 1.81 | 2.04 |
| Fe content | 0.8-1.3 wt % | 2.8-4.7 wt % | Below 350 ppm |

### Cell Adhesion and Structure-Forming Potential of CNT

Cells cultured were incubated with 0.05 mg/ml of single-walled CNT (IL JIN, Korea) for 48 hr at 37° C. in 5% incubator. Then, cell adhesion and structure-forming potential of CNT were analyzed. The cell adhesion property was observed under electron microscope (JEM 200CS, JEOL, Japan). For quantifying the influence of CNT on cell adhesion and the formation of structure, the change of cell morphology was observed with the lapse of time. The numerical values correspond to the number of cells per 1 ml of culture present in the supernatant of centrifuged culture.

### Measurement of Cell Viability

Cells cultured in plate were incubated with 0.05 mg/ml of CNT at 37° C. in 5% incubator. Following the incubation of 12-72 hr, 10 μl of alarmablue solution (Serotec, UK) was added to each well and incubated for additional 3-hr. Reduced alarmablue were measured at 570 nm using ELISA reader (Molecular Devices, Sunnyvale, Calif.) for analyzing the activity of mitochondria. The absorbance background was obtained at 600 nm. The cell viability was calculated with the following formula: Cell viability=[(sample count)−(blank count)/(non-treated control count)−(blank count)]×100 (Shimoke and Chiba, 2001).

### Mac-1 Immunocytochemical Staining

Cells were treated with 50% trypsin, collected and washed with PBS, followed by incubating on slides coated with poly-L-lysine for 1 hr. Cells were washed with PBS, fixed with a mixed solution of acetone/methanol (50%/50%) for 2 min and dried for 5 min. Afterwards, cells were washed three times with PBS, and treated for 10 min with 3% H₂O₂ in ethanol for blocking their inherent peroxidase activities and increasing cell permeability. Cells were incubated with 10% non-immune serum (Zymed Co., USA) for 30 min and with 1:100 diluted primary antibody to Mac-1 (Santacurz, USA) for 2 hr in 37° C. incubator. Cells were washed with PBS and incubated with secondary antibody (biotinylated anti-IgG, VECTA) for 1 hr in 37° C. incubator, followed by developing colorimetric reaction with DAB solution. Cells were further stained with hematoxyline for 10 sec and observed under microscope.

### DCF-DA Staining

10 μM DCFDA (6-carboxy-2′,7′-dichloro-dihydrofluoresceine diacetate, dicarboxymethylester, Invitrogen) dissolved in HCSS buffer (20 mM HEPES, 2.3 mM CaCl₂, 120 mM NaCl, 10 mM NaOH, 5 mM KCl, 1.6 mM MgCl₂ and 15 mM glucose), and 2% Pluronic F-127 surfactant were incubated with cells for 30 min at 37° C. The fluorescence from DCF caused by intracellular free radicals was observed at room temperature under Olympus IX70 inverted microscope (Excitation=488 nm, Emission=510 nm). Images were taken using a CCD camera and analyzed using NIH Image 1.65 program or using flow cytometry (GENios, Tecan, N.C., USA) (Excitation=485 nm, Emission=510 nm).

### Animal Tests

Animal Models with Degenerative Diseases

Animal models having brain ischemia were prepared after adopting 5 week aged male mice and rats (SAMTACO, Korea). Nylon fiber was injected into the right neck artery of mice providing blood to brain and 30-min later, the nylon fiber was removed to resume blood circulation, followed by suturing neck. Afterwards, mice were maintained for 1 week and subjected to several experiments for behavior observation. Furthermore, stem cells or mixtures of CNT and stem cells were injected into the mouse ischemia model using 18-gauge needle. Where only stem cells were injected, 5 μl of suspension (40000 cells/μl) were injected. In the case that a mixture of CNT and stem cells was injected, a mixture of 2×10⁵ cells and 0.02 mg/ml CNT was injected in a volume of 5 μl.

For constructing Alzheimer's animal model, 5-week aged male mice were used. β-amlyloid 1-42 protein (Biosource CA USA) was injected into a region of interest using a stereotaxic system. In this experiment, β-amlyloid proteins were injected into the intraventricular zone. After 1 week after injection, β-amlyloid proteins were re-injected in the same manner. After 2 weeks, the behavior experiments were made and the administration of stem cells or a mixture of CNT and stem cells was done. The dosage of cell therapeutics was the same as the brain ischemia model.

For Parkinson's animal model, the substantial nigra of 5-week aged male mice were injected with 6-OHDA (6-hydroxydopamine) using a stereotaxic system, inducing selective death of dopaminergic neuronal cells in striatum. 2-week later, the behavior experiments were made and the administration of stem cells or a mixture of CNT and stem cells was then done. The dosage of cell therapeutics was the same as the brain ischemia model.

### Injection of Carbon Nanotubes

Where only CNT (f-SWCNT or f-MWCNT) was injected, it was administered in the concentration of 0.2 mg/2 ml. Where CNT was injected together with stem cells, a mixture of 0.2 mg/l ml CNT and 200000 cells/1 ml was administered. Prior to 1 hr of injection, CNT molecules were subjected to ultrasonication to dissociate them.

### Behavior Experiments

### 8-Arm Radial Maze Test

Radial arm maze having eight mazes was used. It has octagonal central platform of which diameter, height and length were 40 cm, 30 cm and 15 cm, respectively. Eight arms (70 cm of length, 9 cm of width and 8 cm of height) are protruded out of the octagonal center. Door are positioned between the central platform and arms, feeding plates (5 cm×5 cm×2.5 cm) for providing compensation are placed at the terminal portion of mazes.

Animals not injected with stem cells, only stem cells or a mixture of stem cells and CNT were subjected to radial maze test. Learning of working memory was determined based on the time period for finding and eating water and the frequency of error per performance. Learning of referencing memory was determined based on the fact that the time period for finding and eating water placed in four mazes and the frequency of error per performance.

Prior to 30 hr of test, mice were deprived of water to induce thirst. On 1^{th} day, all passages to eight mazes were blocked and mice were placed in the central platform for 5 min to be adapted to test environments. From the subsequent days, mice were undergone learning.

Procedures of Learning for Working Memory: 0.1 ml of water was placed at the terminal portion of all eight mazes, mice were placed for 1 min in the central platform for adaptation, and then placed under free accessible environments by opening doors of eight mazes. The first performance was ended when mice visited once all eight mazes and took water, or the time period of test exceeds 5 min. The cases in which mice did not take water in mazes or mice visited twice the same maze were considered as error behavior.

In this test, the most effective performance is that mice visit once each of eight mazes and take water in the 5 min limit. When the error rate was decreased below 5%, learning was considered terminal and test was ended.

Procedures of Learning for Reference Memory: 0.1 ml of water was placed at the terminal portion of four mazes, mice were placed for 1 min in the central platform for adaptation, and then placed under free accessible environments by opening doors of eight mazes. The first performance was ended when mice visited once the four mazes and took water, or the time period of test exceeds 5 min. The cases in which mice did not take water in water-containing mazes or mice visited maze without water were considered as error behavior.

In this test, the most effective performance is that mice visit once each of four mazes with water and take water in the 5 min limit. When the error rate was decreased below 5%, the test was ended.

### Water Maze Test

Spatial learning and memory were tested in accordance with Morris water maze procedure (Morris, R., Developments of a water-maze procedure for studying spatial learning in the rat. J. Neurosci. Methods 11:47-60 (1984)). Animals in each experimental group were trained four times to each of four directions for 5 days to find a platform in a water maze. The time interval of learning was at least 25 min.

### Passive Avoidance Test

Passive avoidance test with stimuli was performed to analyze the acquiring and maintenance of learning and memory. In this test, each animal was trained to move from noisy and light environments to quiet and dark environments. Where mice escape from stimuli and move to dark environments within 20 sec, learning was considered terminal. Prior to providing stimuli, mice were adapted for 180 sec in a stimuli-generating space. Upon providing stimuli, a door was opened to move to dark space without stimuli. The stimuli are noise and light and continue for no more than 90 sec. When mice move to the dark space without stimuli, the door is closed and stimuli are ended. Mice were continuously trained for 4 days in the manner described above. On 5^{th} day, where there are mice moving to the dark space, 0.1 mA stimulus was provided for 3 sec to mice for recognizing mice the fact that the movement to the dark space is associated with electric stimulus. On 2^{nd} day after such shock learning, the period of time for the movement to the dark space was measured in the same manner as learning. At this time, where mice moved to the dark space without stimuli, the electric stimulus was not provided. The shorter period of time for movement was considered to the damage of learning and memory.

### Results

### Cell Adhesiveness of CNT and Influence of CNT on Cell Adhesion

To verify that CNT is adhesive to various cell types such as neuronal cell, cells were treated with CNT and observed from 12 hr to 72 hr. For acting as stem cell scaffold, CNT should adhere to various cell types and induce the aggregation of stem cells. Therefore, cell aggregation and weight change were analyzed.

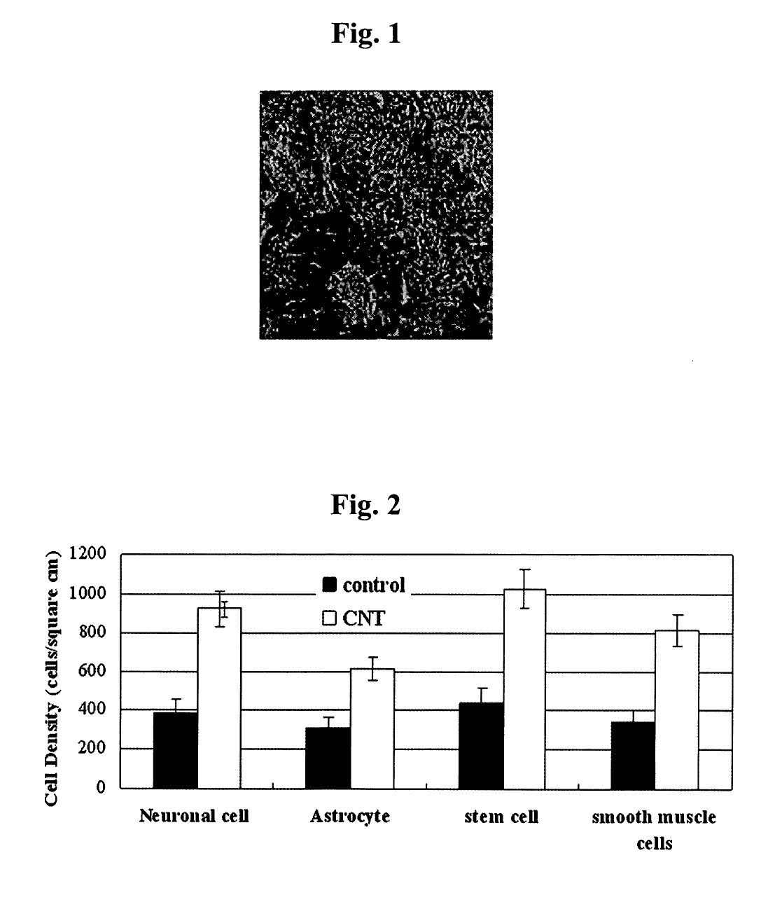

As shown in FIG. 1, electron microscope image, it was revealed that CNT has adhesiveness to P19 EC cells. Furthermore, CNT adhered to neuronal cells such as SK-NSH and astrocytes.

We have tested whether the cell adhesiveness of CNT verified in FIG. 1 affects the adhesion of cells. To elucidate the adhesion of cells in the presence of CNT, the cell density in cell culture dish was measured. As shown in FIG. 2, all cells treated with CNT, in particular, stem cells and neuronal cells exhibit improved adhesion. However, astrocytes were shown less improved adhesion compared to other cell types.

Therefore, it would be recognized that CNT itself has the adhesiveness property to cells and increases cell-to-cell adhesion as well.

### Influence of Carbon Nanotubes on Viability of Microglial Cells

To verify the influence of various carbon nanotubes on microglial cell BV-2 (ATCC), one of mouse brain cells, cells were cultured in DMEM containing 1% FBS prior to the treatment of CNT. The cell viability was measured by alamarBlue assay. This experiment used three type CNT, functionalized Single-Walled CNT (f-SWCNT), functionalized Multi-Walled CNT (f-MWCNT) and CNT fiber in the concentration of 10, 100, 1000 and 10000 μg/ml. The observations were made for 48 hr in the time interval of 12 hr.

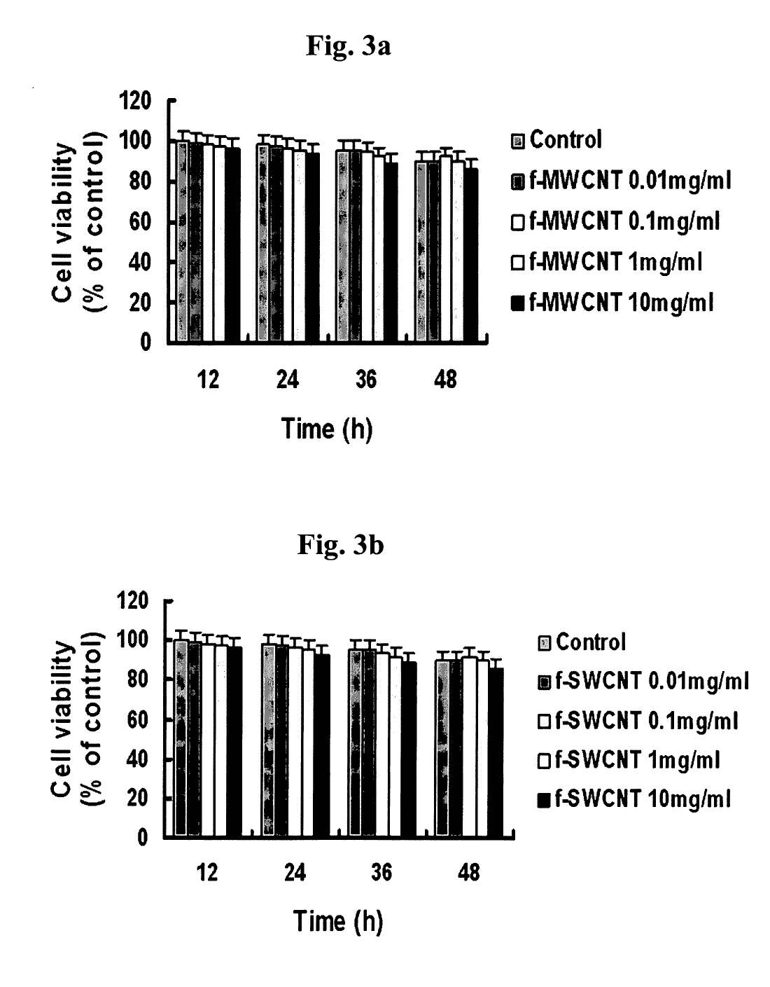

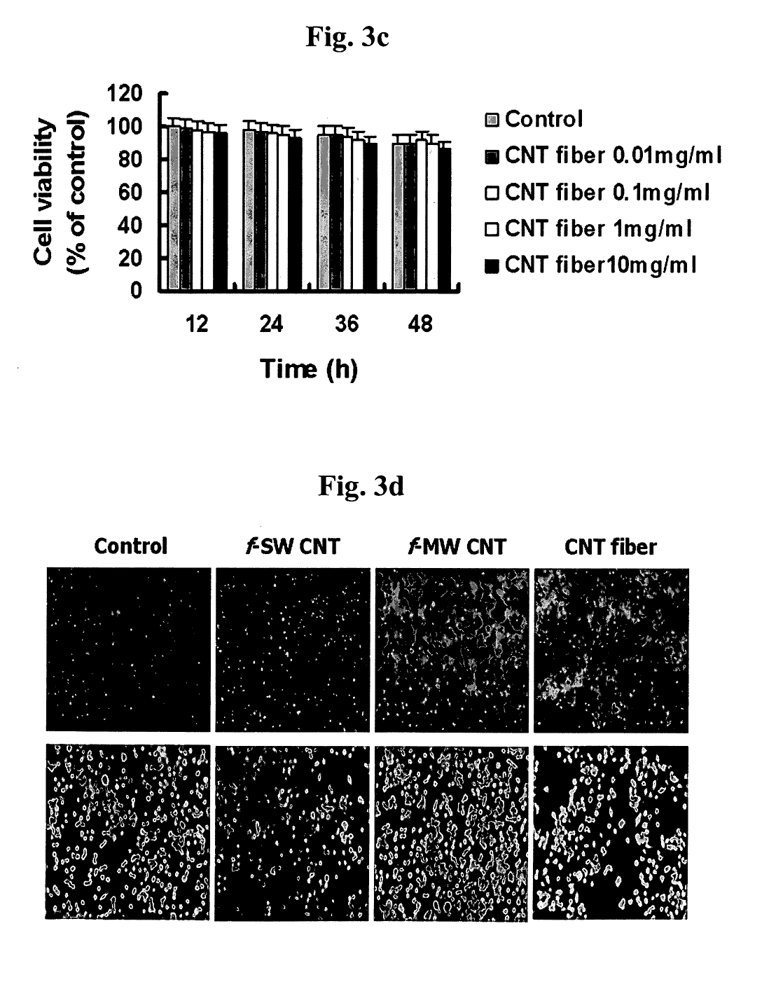

As shown in FIG. 3a, the treatment with f-SWCNT up to 48 hr in the concentration of 10, 100, 1000 and 10000 μg/ml exhibits little or no effect on cell viability. As shown in FIGS. 3a and 3b, f-MWCNT and CNT fiber also exhibit little or no effect on cell viability. In addition, staining with PI and FDA was carried out to observe the occurrence of cell death. The red fluorescent staining with PI indicates dead cells, and the green fluorescent staining with FDA indicates live cells. FIG. 3d represents the results of 48-hr treatments with 10000 μg/ml f-SWCNT, f-MWCNT or CNT fiber. Cells with red fluorescence are scarcely detected.

Summarizing the results, all of f-SWCNT, f-MWCNT and CNT fiber of less than 10000 μg/ml exhibit little or no affect on cell death and viability. Therefore, the safety of carbon nanotubes as scaffold for cell therapy is very marked.

### Carbon Nanotubes and Inflammation in Microglial Cells

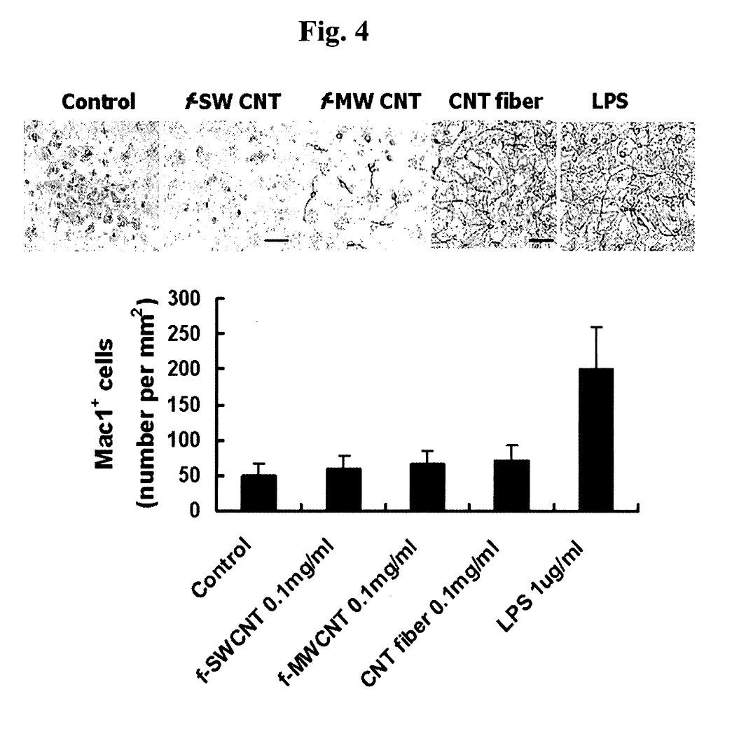

Microglial BV-2 cells were incubated with 100 μg/ml of functionalized Single-Walled CNT (f-SWCNT), functionalized Multi-Walled CNT (f-MWCNT) or CNT fiber for 24 hr. Afterwards, microglial cells showing inflammatory reactions were detected by Mac-1 immunocytochemical staining. As shown in FIG. 4, it was revealed that all of f-SWCNT, f-MWCNT and CNT fiber show little or no different results from control group. Therefore, it would be appreciated that CNT induces no inflammatory reactions. The most right image is a positive control treated with 1 μg/ml LPS. The bottom graph corresponds to the quantified results of Mac-1 immunocytochemical staining.

### Carbon Nanotubes and Reactive Oxygen Species in Microglial Cells

The reactive oxygen species, a main cause of oxidative stresses, affect various signal transduction pathways in cells such as cell death, survival, differentiation and inflammatory reactions. Oxidative stress generated by stimuli or environmental factors has been reported to activate a signal transduction pathway related to inflammatory reactions, triggering downstream signal transduction. Therefore, we tested whether carbon nanotubes cause the generation of reactive oxygen species associated with inflammatory reactions.

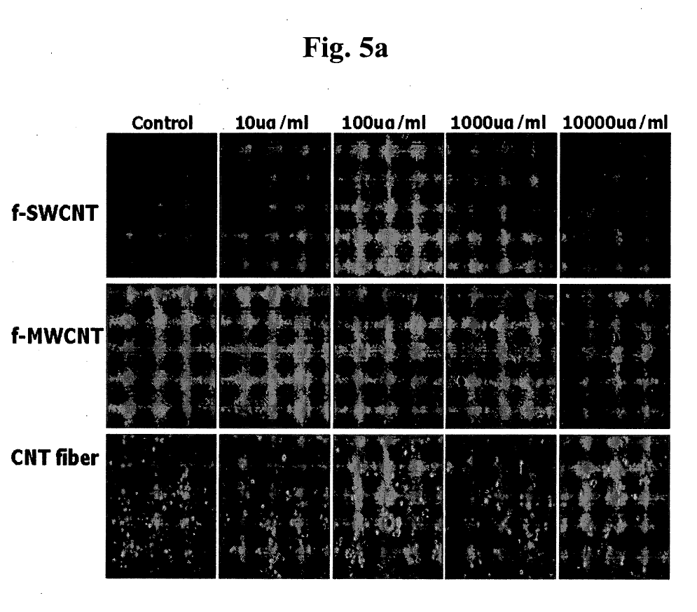

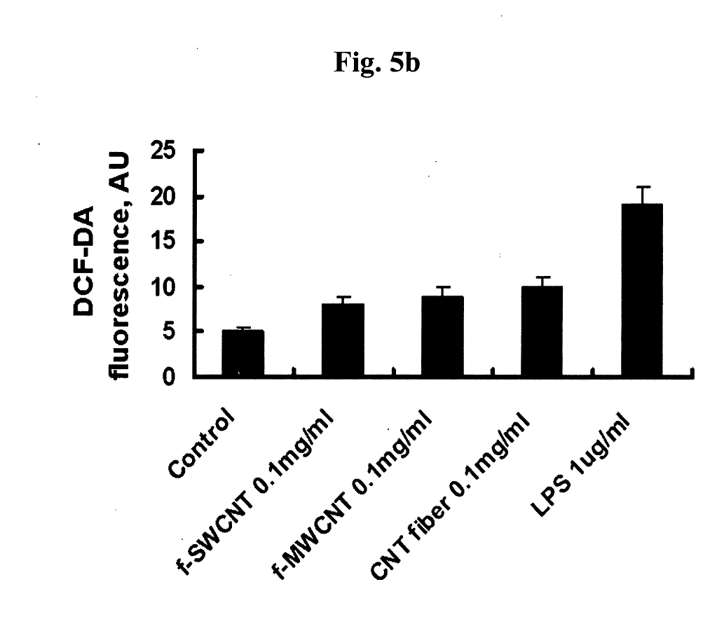

Microglial BV-2 cells were incubated with functionalized Single-Walled CNT (f-SWCNT), functionalized Multi-Walled CNT (f-MWCNT) or CNT fiber in the concentration of 10, 100, 1000 and 10000 μg/ml for 1 hr, 2 hr or 6 hr, and the generation of reactive oxygen species was then analyzed by DCF-DA staining method. As shown in FIG. 5a, all of f-SWCNT, f-MWCNT and CNT fiber cause little or no reactive oxygen species. This result is also confirmed in FIG. 5b corresponding to the quantified results using fluoremeter.

Therefore, it could be understood that CNT molecules exhibit no cytotoxicity and no influence on cell viability, and induce no inflammatory reactions and the generation of reactive oxygen species.

Consequently, these results urge us to reason that CNT molecules are considerably safe materials for stem cell scaffold.

### In Vivo Toxicity of CNT in Mouse Brain

The experiments described previously demonstrate that CNT has no in vitro toxicities. We examined in vivo toxicities of CNT prior to clinical application. CNT (f-SWCNT or f-MWCNT) was directly injected into intraventricular zone of 5-week aged mouse and the viability of mouse was analyzed.

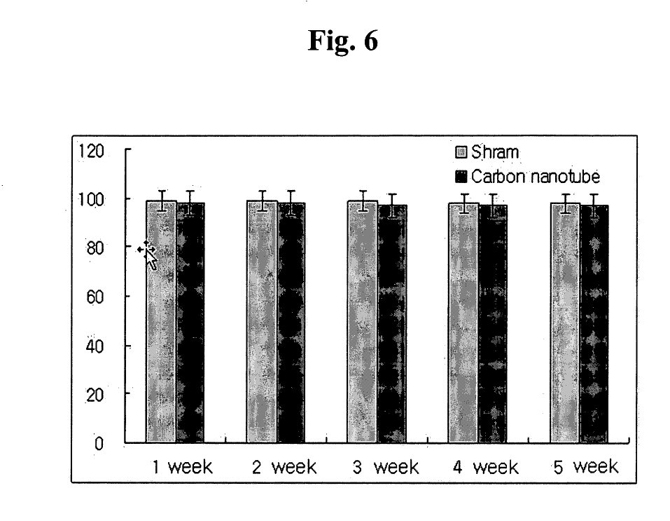

As shown in FIG. 6, the injection of CNT exerts no influence on mouse. In addition, it was also verified by histological staining that the injection of CNT does not result in death and loss of neuronal cells.

This result demonstrates that CNT has no in vivo toxicity and does not cause brain malfunction.

Based on results related to non-toxicity of CNT, we finally evaluate the therapeutic efficacy CNT by transplanting stem cells together with CNT.

### Therapeutic Efficacy of Mixture of Stem Cells and CNT to Parkinson's Disease

The therapeutic efficacy of a mixture of CNT (f-SWCNT or f-MWCNT) and stem cells for Parkinson's disease was evaluated using Parkinson's mouse model prepared with 6-OHDA.

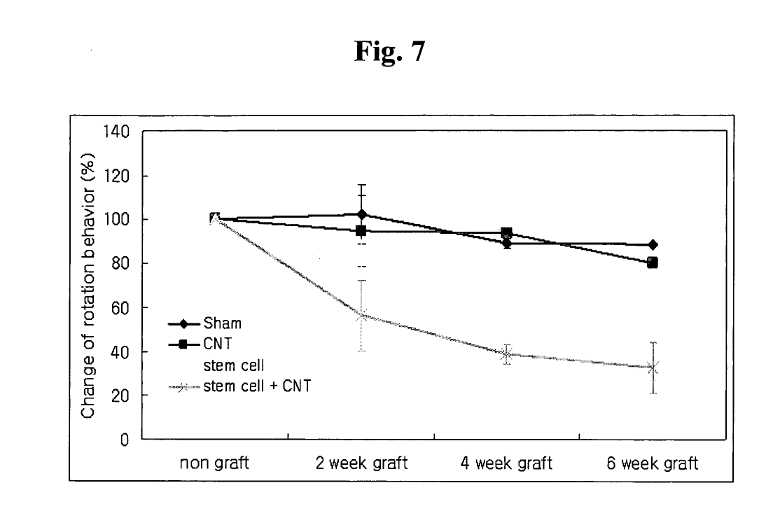

As shown in FIG. 7, mixtures of CNT and stem cells exhibit much better therapeutic efficacy than only stem cells. It could be understood that the improved therapeutic efficacy results from the contribution of CNT to the formation of neuron networking of stem cells.

### Therapeutic Efficacy of Mixture of Stem Cells and CNT in Ischemia Animal Model

For brain ischemia model, we evaluated whether CNT enhances the therapeutic efficacy of stem cells.

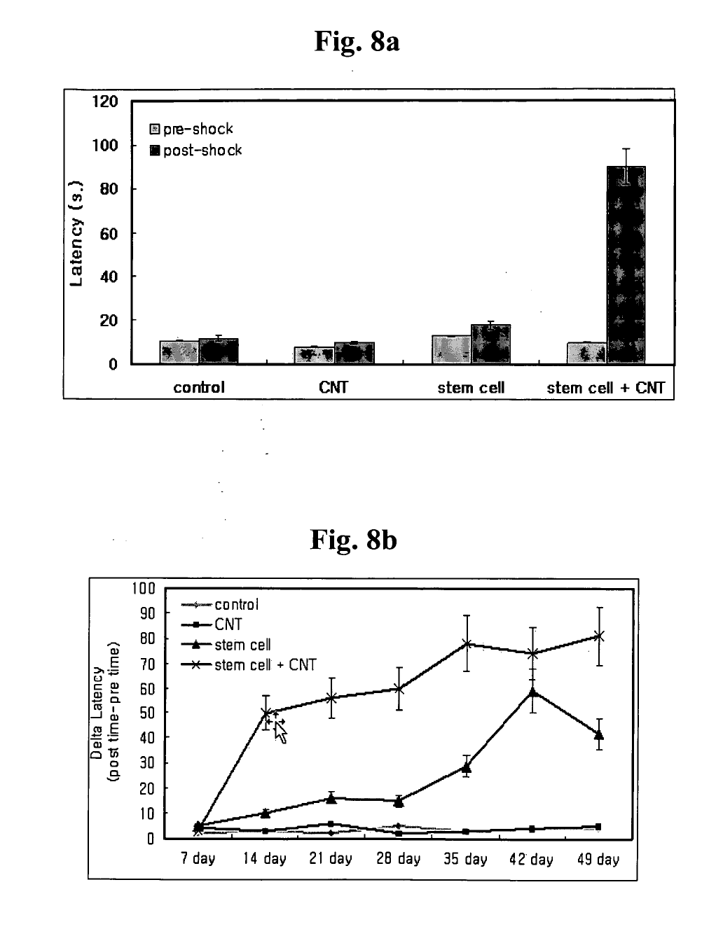

As shown in FIG. 8a, mixtures of CNT and stem cells injected into a brain injury region show much better therapeutic efficacy on behavior abnormality than only stem cells. Furthermore, mixtures of CNT and stem cells exert significant recovery effects on spatial cognition, learning and memory. In FIG. 8a, memory increases as latency time becomes longer.

FIG. 8b represents statistical results of passive avoidance test carried out every week after transplantation. The results demonstrate that the transplantation of stem cells together with CNT in ischemia model gives much more significant treatment in faster manner than a sole transplantation of stem cells. Where only stem cells were administered, the improvement in cognition and learning was observed post 4 weeks of transplantation. However, the transplantation of stem cells together with CNT exhibits significant improvement in cognition and learning within 2 weeks of transplantation. Furthermore, it was also revealed that CNT promotes long-term survival of stem cells, enabling sustained therapeutic efficacies of stem cells to be true.

### Improvement in Behavior and Cognitive Function by Stem Cells and CNT in Alzheimer's Model

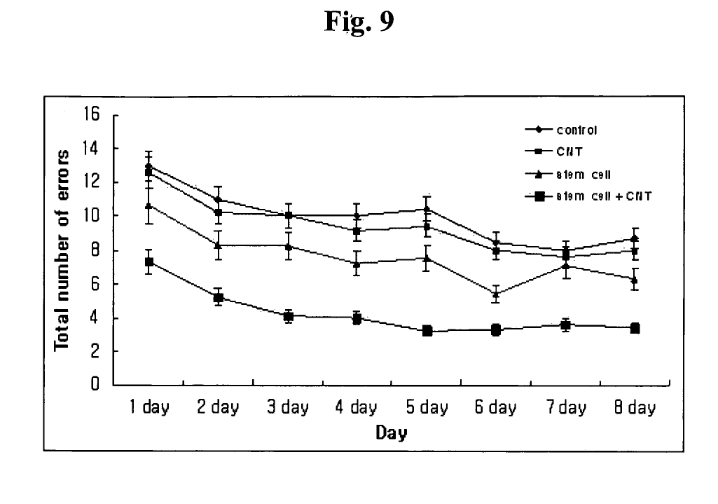

SAT (spatial alteration task) was verified through 8-arm radial maze test using Alzheimer's animal models. As shown in FIG. 9, stem cells along with CNT molecules exhibit much better improvement in memory than only stem cells. Specifically, it was revealed that stem cells along with CNT molecules give stabilized and continuous improvement in memory, which is still better therapeutic efficacy that only stem cells.

As described hereinabove, the present invention provides a non-cytotoxic scaffold for transplanting a stem cell. Furthermore, the present invention provides a composition for stem cell therapy exhibiting improved therapeutic efficacy. The scaffold for transplanting stem cells comprising carbon nanotubes show excellent effects without cytotoxicity on networking between differentiated stem cells and tissues present in transplantation sites, thereby showing significant cell therapy efficacy.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A scaffold for transplanting a stem cell, which comprises a carbon nanotube; wherein the scaffold does not exhibit cytotoxicity.

2. A composition for stem cell therapy, which comprises: (a) a stem cell; and (b) a carbon nanotube serving as a stem cell scaffold without cytotoxicity.

3. The composition according to claim 2, wherein the stem cell is embryonic stem cell or adult stem cell.

4. The composition according to claim 3, wherein the stem cell is neuronal stem cell and the composition is one for treating neuronal diseases.

5. The composition according to claim 4, wherein the neuronal disease is selected from the group consisting of neurodegenerative disorder and ischemia-reperfusion injury.

6. The composition according to claim 5, wherein the neurodegenerative disorder is selected from the group consisting of Alzheimer's disease, Huntington's disease, Parkinson's disease and amyotrophic lateral sclerosis.

7. The composition according to claim 5, wherein the ischemia-reperfusion injury is ischemic stroke.

8. The composition according to claim 2, wherein the carbon nanotube is in the form of suspension.

9. A cell therapy method using a stem cell, which comprises administering to an animal a composition for stem cell therapy comprising (a) a stem cell; and (b) a carbon nanotube serving as a stem cell scaffold without cytotoxicity.

10. The cell therapy method according to claim 9, wherein the stem cell is embryonic stem cell or adult stem cell.

11. The cell therapy method according to claim 10, wherein the stem cell is neuronal stem cell and the composition is one for treating neuronal diseases.

12. The cell therapy method according to claim 11, wherein the neuronal disease is selected from the group consisting of neurodegenerative disorder and ischemia-reperfusion injury.

13. The cell therapy method according to claim 12, wherein the neurodegenerative disorder is selected from the group consisting of Alzheimer's disease, Huntington's disease, Parkinson's disease and amyotrophic lateral sclerosis.

14. The cell therapy method according to claim 12, wherein the ischemia-reperfusion injury is ischemic stroke.

15. The cell therapy method according to claim 9, wherein the carbon nanotube is in the form of suspension.

16. Use of a composition comprising (a) a stem cell; and (b) a carbon nanotube serving as a stem cell scaffold without cytotoxicity for manufacturing a medicament for cell therapy.

17. The use according to claim 16, wherein the stem cell is embryonic stem cell or adult stem cell.

18. The use according to claim 17, wherein the stem cell is neuronal stem cell and the composition is one for treating neuronal diseases.

19. The use according to claim 18, wherein the neuronal disease is selected from the group consisting of neurodegenerative disorder and ischemia-reperfusion injury.

20. The use according to claim 19, wherein the neurodegenerative disorder is selected from the group consisting of Alzheimer's disease, Huntington's disease, Parkinson's disease and amyotrophic lateral sclerosis.

21. The use according to claim 19, wherein the ischemia-reperfusion injury is ischemic stroke.

22. The use according to claim 16, wherein the carbon nanotube is in the form of suspension.
